(19) United States
(12) Patent Application Publication
(10) Pub. No.: US 2008/0166330 A1
(43) Pub. Date: Jul. 10, 2008

(54) Bacterial Compositions For Prevention Or Treatment Of Atherosclerotic Disorders

(76) Inventors: Ivan Petyaev, Cambridge (GB); Andrea Pfeifer, Lausanne (CH)

Correspondence Address: NIXON & VANDERHYE, PC, 901 NORTH GLEBE ROAD, 11TH FLOOR, ARLINGTON, VA, 22203, US

(21) Appl. No.: 11/666,726

(22) PCT Filed: Nov. 1, 2005
(86) PCT No.: PCT/GB2005/004215

§ 371 Date: May 2, 2007

(30) Foreign Application Priority Data
Nov. 5, 2004 (GB) 0424552.8

(51) Int. Cl. A61K 35/74; A61P 9/10

(52) U.S. Cl. 424/93.44; 424/93.4; 424/93.45

(57) ABSTRACT

This invention relates to the treatment of atherosclerotic conditions through the inhibition of lipid oxidising abzymes by altering the composition of the intestinal microbial flora, for example by administering a bacterial composition to the individual. Suitable bacterial compositions may comprise, for example enteric bacteria such as lactobacilli spp, bifidobacteria spp, streptococcal spp and E. coli.

This invention relates to methods and means for the treatment of atherosclerotic conditions.

Atherosclerosis is a complex, multi-factor condition that is characterised by the formation of atherosclerotic lesions within arteries. Atherosclerotic lesions generally consist of lipid engorged monocytes and macrophages (foam cells) covered by a fibrous cap. These lesions lead to narrowing and hardening of arteries and are associated with a range of cardiovascular diseases. The interaction of a range of different cells is important for the development of atherosclerotic lesions including, monocyte/macrophages, T lymphocytes, dendritic cells, endothelial cells and smooth muscle cells. Other important factors for the development of these lesions in the arterial wall are the extracellular matrix, its proteoglycans (biglycan and versican), and collagen. Various serum lipoproteins have also be associated with atherosclerosis, including anti-LDL immune complexes, oxidized low density lipoprotein (LDL), remnant lipoprotein (beta-VLDL), lipoprotein [Lp] (a), apolipoproteins apoA-I, apoB, and apoE.

Lipid oxidising antibodies (‘abzymes’) have been identified as a key pathogenic factor in atherosclerotic conditions and the presence of elevated levels of these abzymes in the serum is known to be indicative of the onset of atherosclerotic conditions, including cardiovascular conditions such as coronary heart disease (WO03/019196, WO03/017992 and WO03/019198).

The present inventor has recognised that the intestinal microbial flora of patients with atherosclerotic conditions is distinct from that of healthy individuals and that this contributes to the atherosclerotic condition. Bacterial compositions are shown herein to directly inhibit abzyme activity, and such compositions are also shown to be useful in treating atherosclerotic conditions in patients and in preventing or delaying the onset or recurrence of such conditions, for example by altering the composition of the intestinal microbial flora.

One aspect of the invention provides a method of treating an atherosclerotic disorder in an individual comprising:
administering a bacterial composition to the individual.

Related aspects provide a bacterial composition for use in treating an atherosclerotic disorder in an individual and the use of a bacterial composition in the manufacture of a medicament for use in the treatment of an atherosclerotic disorder.

A suitable bacterial composition may comprise live or viable bacteria cells which are capable of growth and division in the digestive tract of the individual after administration. Suitable bacterial cells include so-called ‘probiotics’ which improve, restore or maintain the microbial balance of the intestinal microflora of the individual (Fuller R: Probiotics in Man and Animals, J Appl. Bacteriol 1989; 66: 365-365-378 and Havenaar R, Brink B, Huis In't Veld JHJ: Selection of Strains for Probiotic Use. In Scientific Basis of the Probiotic Use, ed. R. Fuller, Chapman and Hall, London UK, 1992).

Preferred bacteria include enteric bacteria i.e. bacteria which form part of the intestinal microflora of a healthy individual.

Preferably, a bacterial composition has abzyme inhibition activity i.e. the composition inhibits the lipid oxidation activity of serum antibodies. The inhibition of abzyme activity may be determined using known techniques (see for example WO03/019196, WO03/017992 and WO03/019198), which are described in more detail below.

A suitable bacterial composition may comprise one, two, three, four or five or more different species of bacteria. For example, a suitable composition may include one or more -of non-pathogenic E. coli, Bifidobacteria spp, Streptococcal spp and lactic acid producing bacteria such as Lactobacilli spp.

Suitable Lactobacilli spp include L plantarum, L. reuteri, L. bulgaricus, Lactobacillus GG, L. acidophilus, L. casei, L. fermentum, L. gasseri, L. johnsonii, L. lactis, L. paracasei L. plantarum, L. rhamnosus, L. salivarius and related Lactobacillus spp (Gilliland S E Micro Rev. 1990; 87; 175-188; Gorbach S L: 1990; 22-37-41)

Suitable Bifidobacteria spp include B. bifidum, B. breve, B. lactis, B. longum and B. infantis.

Suitable Streptococcal spp include S. thermophilus.

Other examples of suitable bacterial species are set out in Table 3.

Preferably, the bacterial composition comprises one or more Lactobacilli spp. For example, a suitable bacterial composition may comprise L. acidophilus, L. casei casei, and L. casei rhamnosus.

A bacterial composition suitable for use in the present methods may be prepared by combining a starter culture comprising bacterial cells of the selected species in a carbohydrate enriched media. The starter culture may comprise viable bacteria cells. The combination of starter culture and media may then be incubated under controlled conditions. During the incubation, the temperature and pH of the culture may be monitored and controlled as is well known in the art.

The process may be halted when a desired ratio of different bacterial species and a desired total concentration of organisms has been reached.

In some embodiments, bacteria may be packaged for administration directly after fermentation. In other embodiments, the bacteria in the culture medium may be concentrated and then lyophilised, freeze dried or air dried using standard methods, prior to packaging.

After concentrating and lyophilizing, the bacterial composition can be packaged into desired dosing units. The packaged dosing units may be in any convenient form, including for example packets, capsules, caplets, or tablets. The concentrated and lyophilised bacteria in some embodiments may be compounded with other foodstuffs.

A bacterial composition suitable for use in the present methods may comprise any amount of bacterial cells which is sufficient to produce a therapeutic effect. For example, a composition may comprise 10⁴ to 10¹⁰ cells, for example, 10⁵, 10⁶, 10⁷, 10⁸ or 10⁹ cells.

The bacterial composition may be a liquid, solid, or semi-solid. For example, the bacterial composition may be administered orally as a bolus in the form of a gelatin capsule, pressed tablet, or gel cap or in the form of a paste or liquid.

The bacterial composition may be compounded with additional ingredients, such as flavour enhancers, sweeteners, viscosity enhancers and other food additives. Additional ingredients may include carbohydrate polymers comprising one or more of the group consisting of trehalose, glucose, sucrose, fructose and maltose, proteins such as albumin and/or whey, and other supplements such as L-glutamine and N-acetyl glucosamine. Food additives may include conventional food supplement fillers and extenders such as, rice flour.

Additional ingredients may also include therapeutic agents, including, for example, abzyme inhibitors as described herein.

The bacterial composition is preferably administered in a “prophylactically effective amount” or a “therapeutically effective amount” (as the case may be, although prophylaxis may be considered therapy), this being sufficient to show benefit to the individual. The actual amount of bacteria administered will depend on the nature and severity of what is being treated.

Conveniently, the composition may be administered orally at any suitable dosage, for example a dosage ranging from about 100 milligrams to about 800 milligrams of bacteria (dry wt) per day. Preferably, the dosage ranges from about 200 milligrams to about 400 milligrams per day.

An atherosclerotic condition suitable for treatment as described herein may include any cardiovascular condition which is associated with the formation and accumulation of fatty deposits in the vessels of the cardiovascular system or is a consequence of such formation and accumulation.

Atherosclerotic conditions include atherosclerosis; heart diseases such as ischaemic (coronary) heart disease, myocardial ischaemia (angina) and myocardial infarction; and other cardiovascular disorders such as aneurismal disease, atheromatous peripheral vascular disease, aortoiliac disease, chronic and critical lower limb ischaemia, visceral ischaemia, renal artery disease, cerebrovascular disease, stroke, atherosclerotic retinopathy, thrombosis, aberrant blood clotting and hypertension. Such conditions may be medical or veterinary conditions.

Individuals which may be treated as described herein include humans and non-human animals. References to ‘human’ herein should be understood to include ‘non-human animal’, unless context dictates otherwise.

An individual may be assessed for an atherosclerotic condition before, during or after treatment with a bacterial composition as described herein by determining the presence or level of abzymes in the serum of the individual.

Abzymes are catalytic antibodies which bind and oxidise lipids and lipoproteins to generate atherogenic factors. Abzymes may be anti-Chlamydia abzymes i.e. they may bind or be reactive with Chlamydia cells, for example Chlamydial cells from a species belonging to the Chlamydia psittaci group such as Chlamydia psittaci and Chlamydia pneumoniae.

The presence of abzymes in the serum is indicative that an individual is suffering from or at risk of an atherosclerotic condition.

In some embodiments, serum abzyme activity may be reduced or eliminated prior to administration of the bacterial composition.

Serum abzyme levels may be reduced or eliminated, for example, by administration of an abzyme inhibitor.

An abzyme inhibitor is a molecule which reduces or inhibits the lipid oxidising activity of an abzyme. Abzyme inhibitors may include metal chelators, examples of which are shown in Table 2. Metal ions of transient valence at the catalytic centre of an abzyme are specifically targeted by such chelators to neutralise the catalytic properties of the abzyme. Metal chelators that inhibit abzymes include aspirin.

Other such inhibitors include substrate antagonists which prevent binding of the abzyme with its target epitope(s) in the organism of the host. Binding antagonists may be either be peptide, lipid, polysaccharide, or any other synthetic or naturally occurring product which imitates an epitope of the abzyme. An antagonist may be modeled on a lipid antigen, which may for example be a host, i.e. a human antigen or a pathogen antigen. Inhibitors which block the binding sites of the abzymes include anti-idiotypic antibody molecules, including Fab/Fv or other antibody fragments and derivatives, or peptide molecules presenting the fragment(s) of the complementary loop of their active centres (i.e. which imitate the anti-idiotypic antibody molecule) which would enable them to inhibit the binding of the abzymes with their target antigens. Suitable antibody molecules may be made by using a polyclonal or monoclonal strategy or by phage display.

The anti-bacterial drug azithromycin has been shown to inhibit abzyme activity. Inhibitors may include both molecules structurally related to azithromycin and anti-microbial agents such as erythromycin, roxithromycin, ofloxacin, clinafloxacin, ciprofloxacin, clindamycin, azithromycin, doxycycline, minocycline and tetracycline.

Other examples of abzyme inhibitors include desferrioxamine mesylate, haem derivatives, penicillamine, tiopronin, trientine dihydrochloride, diethyldithiocarbamate, disodium/trisodium edetate, acetylsalicylic acid, edetic acid, unithiol, tocopherols, mannitol, silidianin, catechins, such as (−)-epigallocatechin gallate (EGCG), (−)-epicatechin gallate (ECG), (−)-epigallocatechin (EGC) and (−)-epicatechin (EC), and ascorbic acid.

Serum abzyme levels may also be reduced by modifying the active centre of an abzyme to inactivate its catalytic properties. For example, the active centre of an abzyme may contain a photo-(UV-)sensitive group(s) that may be modified by an extra-corporal (UV) irradiation of plasma/serum to inactivate the lipid peroxidation properties of these molecules.

Following treatment to reduce or eliminate serum abzyme activity, levels of serum abzyme activity may be determined for the individual, for example by testing the ability of an antibody from a sample of serum obtained from the individual to oxidise lipid.

Abzyme activity may be determined by measuring or detecting (for example by measuring or detecting) the oxidation of lipid, which may be lipid from the sample, lipid from a foreign antigen such as a Chlamydia cell, or lipid from another source, which may, for example, be added as part of an assay method.

Many methods for determining lipid oxidation and peroxidation are known in the art. Suitable methods are, for example, described in CRC Handbook of Methods for Oxygen Radical Research, CRC Press, Boca Raton, Fla. (1985), Oxygen Radicals in Biological Systems. Methods in Enzymology, v. 186, Academic Press, London (1990); Oxygen Radicals in Biological Systems. Methods in Enzymology, v. 234, Academic Press, San Diego, New York, Boston, London (1994); and Free Radicals. A practical approach. IRL Press, Oxford, New York, Tokyo (1996)

Oxidation may be determined by determining the production or accumulation (i.e. the presence or amount) of a lipid oxidation product or by-product. Oxidation products and/or intermediates of the lipids in which oxidation was initiated may be determined or oxidation products and/or intermediates may be determined of lipids in which oxidation is propagated.

Suitable lipid oxidation products may include aldehydes such as malondialdehyde (MDA), (lipid) peroxides, diene conjugates or hydrocarbon gases. Lipid oxidation products may be determined by any suitable method. For example, lipid peroxidation products may be determined using HPLC (Brown, R. K., and Kelly, F. J In: Free Radicals. A practical approach. IRL Press, Oxford, New York, Tokyo (1996), 119-131), UV spectroscopy (Kinter, M. Quantitative analysis of 4-hydroxy-2-nonenal. Ibid. 133-145), or gas chromatography-mass spectrometry (Morrow, J. D., and Roberts, L. J. F₂-Isoprostanes: prostaglandin-like products of lipid peroxidation. Ibid. 147-157). Conveniently, the production of malondialdehyde (MDA) may be determined by measuring absorbance at an appropriate wavelength such as 525 nm, following reaction with 2-thiobarbituric acid (conveniently 1 mM).

In other embodiments, lipid oxidation may be determined by measuring the disappearance or consumption of substrates such as non-modified lipids or co-substrates such as oxygen.

In addition to testing the ability of serum antibodies from an individual to oxidise lipid, the antibodies may also be tested for ability to bind to a Chlamydia cell. Antibody binding may be determined using any one of a range of standard techniques.

An individual identified as having low or undetectable serum abzyme levels (i.e. abzyme negative) may be treated as described herein to prevent or delay the onset of an atherosclerosis condition.

Another aspect of the invention provides a method of preventing, delaying or reducing the risk of recurrence of an atherosclerotic condition in an individual comprising:
administering a bacterial composition to the individual.

Related aspects provide a bacterial composition for preventing, delaying or reducing the risk of the onset of an atherosclerotic condition and the use of a bacterial composition in the manufacture of a medicament for preventing, delaying or reducing the risk of recurrence of an atherosclerotic condition in an individual.

The individual may have a history of atherosclerotic disorders and/or a history of abzymes in the serum (i.e. a history of being abzyme positive).

The individual may have been previously treated for an atherosclerotic disorder. For example, therapeutic intervention may have eliminated or reduced the abzymes from the serum, for example as described above, prior to administration of the bacterial composition.

Abzyme activity in the serum of the individual may be determined prior to administering the bacterial composition as described herein. In some preferred embodiments, the individual has no serum abzyme activity (i.e. is abzyme negative) when the composition is administered.

The bacterial composition may be administered in a single dose or may be administered periodically over a course of time, for example weekly, monthly, quarterly or annually. The rate and time-course of administration will depend on the nature and severity of the condition and may be determined by a medical practitioner.

Abzyme levels may be monitored periodically in the individual to confirm the efficacy of the treatment.

Control experiments may be performed as appropriate in the methods described herein. The performance of suitable controls is well within the competence and ability of a skilled person in the field.

The disclosures of all documents mentioned herein are incorporated herein by reference.

Various further aspects and embodiments of the present invention will be apparent to those skilled in the art in view of the present disclosure.

Certain aspects and embodiments of the invention will now be illustrated by way of example and with reference to the tables described below.

Table 1 shows the effect of probiotic intestinal bacteria on the lipid oxidising activity of anti-Chlamydia abzymes.

Table 2 shows examples of abzyme inhibitors.

Table 3 shows examples of probiotic bacteria.

Table 4 shows the spectrum of Intestinal Flora in patients with CHD.

Table 5 shows clinical efficacy after 8 weeks treatment of combined therapy of lactobacilli culture with azithromycin on patients with Coronary Heart Disease, CHD, in Phase IIa.

Table 6 shows normalisation of clotting time of combined therapy of Lactobacilli culture with Azithromycin in patients with CHD.

Table 7 shows one-year follow-up of patients with CHD after combined therapy of lactobacilli culture with azithromycin.

### EXPERIMENTAL

Assay for Abzyme Activity

The abzyme activity in a serum sample was determined by diluting the sample 1:1 with 0.05M acetate buffer pH 4.0 to bring the final pH of the sample to between 5.6-5.8.

990 μl of the diluted serum sample was mixed with 10 μl of the commercial live ovine Chlamydia vaccine and the sample incubated overnight (12-16 hours) at 37° C.

250 μl of 40% trichloroacetic acid and 250 μl of 1 mM 2-thiobarbituric acid were then added to each sample and the samples placed in a water bath and boiled for 30 minutes.

The samples were then cooled down, centrifuged at 3,000 g for 10 minutes and the supernatants collected. Absorption of the supernatants was measured at λ 525 nm to determine the concentration of malondialdehydes (MDA) which are products of lipid peroxidation.

Intestinal Bacteria

Samples of intestinal bacteria were obtained from 45-60 year old CHD patients, clinically healthy age adjusted control subjects and healthy 20-25 year old subjects.

The amount of different bacterial species present in the samples were measured using standard microbiological techniques and the results set out in Table 4.

Differences between the healthy and age control subjects indicates that the microbial flora of the intestine alters with increasing age. In particular, the level of “useful” bacteria, such as Lactobacilli, is 100-1,000 lower in older people than in younger people. Significantly, the intestinal microbial flora of CHD patients appears to have a different composition from that of age related controls. In particular, there is an increase in pathogenic microbes such as S. aureus and Candida spp and a reduction in non-pathogenic microbes such as E. coli and lactobacillus spp in CHD patients.

In Vitro Abzyme Inhibition

Abzyme positive human serum was obtained from patients with Coronary Heart Disease (CHD). The presence of abzymes was confirmed as described above.

Lyophilised live bacteria cultures were re-suspended in PBS. Different concentrations of the filtrated solutions of bacteria were added to abzyme positive human serum.

The effects of these solutions on the activity of abzymes were compared with control samples and the results are shown in Table 1.

Lactobacillus spp were observed to directly inhibit lipid-oxidising abzymes in the serum samples.

In Vivo Abzyme Inhibition

A Phase IIa trial was conducted of the effect of combined therapy of lactobacilli culture with azithromycin on patients with Coronary Heart Disease, CHD.

A group of 30 patients with CHD were selected for experimental therapy to reduce/eliminate the activity of anti-Chlamydia abzymes in their serum (the therapy group)). The trial took place in Saratov Cardiological Centre (Russian Federation) from June until August 2002.

The therapy group comprised 23 male and 7 female patients with an average age of 55±1.1 years. Each patient gave written consent for his/her participation in the trial.

All patients had angina of II-III class of Canadian Cardiological Society classification. 15 patients in the therapy group had a history of myocardial infarction in the past year. CHD diagnosis for the other 15 patients in the first group was confirmed by coronary angiography, which detected 70% or more of arterial stenosis.

The progression of the clinical condition of the patients was monitored by the use of the modified Bruce Protocol for treadmill exercise/stress ECG testing and on the Rose-Blackburn Questionnaire (Cardiovascular Survey Methods. WHO, Geneva, 1968).

Patients were daily administered 500 mg azithromycin and 36 mg of a lactobacilli culture (providing about 2×10⁹ cells) comprising L. acidophilus, L. casei casei and L. casei rhamnosus.

After 8 weeks, the clinical efficacy of the combined therapy of lactobacilli culture with azithromycin was observed in CHD patients relative to healthy controls (Table 5) and clotting time was normalised (Table 6).

Treatment for 8 weeks was observed to inhibit abzymes for 3-6 months with clinical benefits still apparent after one year (Table 7).

The observed clinical improvement permitted bypass surgery for 6 patients in the treated group. In the control group only for 1 patient was it possible to do this operation.

The combined therapy reduced the number of both fatal and non-fatal myocardial infarctions and alleviated symptoms of angina (clinical scores based on Rose G., Blackburn H. Questionnaire). For 7 of the patients no apparent symptoms were registered during the observation period.

The above results show that the combined therapy of lactobacilli culture with azithromycin was more beneficial for patients with CHD than previously reported treatments using azythromicin alone.

TABLE 1

| | ID₅₀, in number of | ID₅₀, in protein |
|---|---|---|
| Bacteria | live bacteria | concentration |
| Lactobacilli Culture | | |
| L. acidophilus | 1 ± 0.3 × 10⁴ | 9.4 ± 2.7 μg |
| L. casei casei | | |
| L. casei rhamnosus | | |
| Bifidobacterium | >2 ± 0.4 × 10⁶ | >4.2 ± 0.8 mg |
| Culture | | |

TABLE 2

| Metals | Chelators | Proprietary Preparations |
|---|---|---|
| Fe⁺²/Fe⁺³ | Desferrioxamine | Canad.: Zinecard; Fr.: Cardioxane; |
| | Mesylate | Ital.: Cardioxane; Eucardion; USA: |
| | | Zinecard. |
| | Haem Derivatives | Austral.: Panhematin; Fr.: Normosang; |
| | | USA: Panhematin. |
| Cu⁺¹/Cu⁺² | Penicillamine | Aust.: Artamin; Distamine; Austral.: |
| | | D-Penamine; Belg.: Kelatin; Canad.: |
| | | Cuprimine; Depen; Fr.: Trolovol; |
| | | Ger.: Metacaptase; Trisorcin; |
| | | Trolovol; Irl.: Distamine; Ital.: |
| | | Pemine; Sufortan; Neth.: Cuprimine, |
| | | Distamine; Gerodyl; Kelatin; Norw.: |
| | | Cuprimine; S.Afr.: Metaalcaptase; |
| | | Spain: Cuprein; Sufortanon; Swed.: |
| | | Cuprimine; Switz.: Mercaptyl; UK: |
| | | Distamine, Pendramine; USA: |
| | | Cuprimine; Depen. |
| | Tiopronin | Fr.: Acadione; Ger.: Captimer; Ital.: |
| | | Epatiol; Mucolysin; Mucosyt; Thiola; |
| | | Tioglis; Spain: Sutilan; Switz.: |
| | | Mucolysin; USA: Thiola. |
| | | Multi-ingredient: Ital.: Mucolysin |
| | | Antibiotico; Spain: Hepadigest. |
| | Trientine | USA: Syprine. |
| | Dihydrochloride | |
| | Diethyl- | |
| | dithiocarbamate | |
| | Acetylsalicylic acid | |
| Me⁺²* | Disodium/Trisodium | Fr.: Chelatran; Tracemate; Irl.: |
| | Edetate | Limclair; UK: Limclair; USA: |
| | | Disotate; Endrate. |
| | | Multi-ingredient: Canad.: Murine |
| | | Supplement Tears; Fr.: Vitaclair; |
| | | Ger.: Complete; Duracare; Oxysept; |
| | | UK: Uriflex G; Uriflex R. |
| | Edetic Acid | Multi-ingredient: Ital.: Conta-Lens |
| | | Wetting; USA: Summer'sEve Post- |
| | | Menstrual; Triv; Vagisec Plus; |
| | | Zonite. |
| | Unithiol | Ger.: Dimaval; Mercuval. |

*Any bivalent metal

TABLE 3

| Bacterial Name | Strain Number |
|---|---|
| Bifidobacterium. Adolescentis | 248 UNSW 509400 |
| Bif. bifidum | 248 UNSW 509800 |
| Bif. longum | 248 UNSW 509700 |
| Bif. Pseudolongum | 248 UNSW 509500 |
| Bif. Infantis | 248 UNSW 510000 |
| Bacteroides fragilis | NCTC 9343 |
| Bact. vulgatus | 1ATCC 8482 |
| Lactobacillus viridescens | 1ATCC 12706 |
| L. casei | 1ATCC 25302 |
| L. acidophilus | 1ATCC 4356 |
| L. plantarum | 1ATCC 8014 |
| L. casei subsp. Rhamnosus | 1ATCC 7469 |
| L. fermentum | 1ATCC 9338 |
| L. brevis | 248 UNSW 055100 |
| L. salivarius | 1ATCC 11741 |

TABLE 4

| | Healthy | | |
|---|---|---|---|
| | control, | | |
| | 20-25 | 45-60 years old | |
| Intestinal | years | Age control group, | Patients with CHD, |
| Bacteria | old | n = 6 | n = 11 |
| E. coli, total × 10⁶/g | 300-400 | 100, 2, 300, 410, | 40, 100, 200, 80, |
| | | 310, 400 | 410, 410, 60, 4, 80, |
| | | 2/6 = 33% below | 100, 405 |
| | | healthy control | 8/11 = 73% below |
| | | | healthy control |
| Cocci, % from the | ≦25% | 54%, 76%, 5%, | 1%, 54%, 58%, 7%, |
| total bacteria | | 0.4%, 5%, 1% | 48%, 67%, 8%, 4%, |
| pool | | 2/6 = 33% above | 43%, 62%, 14% |
| | | healthy control | 6/11 = 55% above |
| | | | healthy control |
| Streptococcus | 0 | 0, 0, 0, 0, 0, 0, 0 | 0, 0, 10⁴, 0, 0, |
| aureus | | | 0, 10³, 0, 10⁴, 0, 0 |
| | | | 3/11 = 21% have |
| | | | pathogenic cocci |
| Candida | 0 | 0, 0, 0, 0, 0, 0, 0 | 0, [+], 0, 0, 0, [+], |
| | | | 0, 0, 0, [+], 0 |
| | | | 3/11 = 21% have |
| | | | Candida |
| Bifidobacterium, | 1.0 | 10⁴, 10⁴, 10⁴, 10⁷, | 10⁴, 10⁷, 10⁴, 10⁴, |
| ×10⁷-10⁸/g | | 10⁴, 10³ | 10⁴, 10³, 10⁴, 10⁷, |
| | | 5/6 = 83% below | 10⁴, 10⁴, 10⁴ |
| | | healthy control | 9/11 = 82% below |
| | | | healthy control |
| Lactobacillus, | 1.0 | 10⁴, 10⁷, 10⁴, 10⁷, | 10⁴, 10⁷, 10⁴, 10⁴, |
| ×10⁶-10⁷/g | | 10⁴, 10³ | 10⁴, 10³, 10⁴, 10⁴, |
| | | 4/6 = 67% below | 10⁴, 10⁴, 10⁴ |
| | | healthy control | 10/11 = 91% below |
| | | | healthy control |

TABLE 5

Patients with CHD
n = 30

| | Healthy | Before | 8 weeks after |
|---|---|---|---|
| Parameter | Control | treatment | treatment |
| Abzymes | 6.36 ± 1.14 | 54.9 ± 7.22 | 4.1 ± 1.18 |
| activity | | | p < 0.001 |
| in μM MDA/ml | | | |
| Clinical | 0 | 19.4 ± 0.69 | 14.9 ± 0.67 |
| Status | | | p < 0.001 |
| Rose G. - | | | |
| Blackburn H. | | | |
| Questionnaire | | | |

TABLE 6

Patients with CHD,
n = 30

| Parameters of | Healthy | Before | 8 weeks after |
|---|---|---|---|
| Coagulation* | Control | treatment | treatment |
| APTT | 49.1 ± 7.00 | 22.4 ± 0.89 | 46.9 ± 6.45 |
| | | | p < 0.005 |
| PT | 23.7 ± 4.01 | 13.5 ± 0.94 | 25.3 ± 4.05 |
| | | | p < 0.05 |
| SCT | 248 ± 10.0 | 151 ± 15.0 | 235 ± 17.9 |
| | | | p < 0.005 |
| KCT | 133 ± 23.7 | 51.2 ± 4.59 | 126 ± 34.2 |
| | | | p > 0.05 |

*APTT—Activated Partial Thromboplastin Time, PT—Prothrombin Time, SCT—Silica Clotting Time, KCT—Kaolin Clotting Time, in sec.

TABLE 7

| Clinical | | | | |
|---|---|---|---|---|
| condition | | | | |
| and events for | Control | | Treatment | |
| the period of | n = 20 | | n = 29 | |
| observation | June 2002 | June 2003 | June 2002 | June 2003 |
| Clinical scores | 19.8 ± 1.43 | 22.0 ± 2.15 | 19.4 ± 0.69 | 11.3 ± 0.50 |
| based on | | p > 0.05 | | p < 0.001 |
| Rose G., | | | | |
| Blackburn H. | | | | |
| Questionnaire | | | | |

| | | |
|---|---|---|
| Coronary | 1 (5%) | 6 (21%) |
| bypass | | |
| surgery | | |
| CHD death | 4 (20%) | 2 (7%) |
| Non-fatal | 4 (20%) | 1 (3%) |
| infarction | | |
| No apparent | 0 | 7 (24%) |
| symptoms | | |
| of angina | | |

1. A method of treating an atherosclerotic disorder in an individual comprising: administering a bacterial composition to the individual.

2. A method according to claim 1 comprising reducing the level of abzyme activity in the cardiovascular system of said individual prior to administering the bacterial composition.

3. A method according to claim 2 wherein the level of abzyme activity is reduced by administration of an abzyme inhibitor

4. A method according to claim 3 wherein the abzyme inhibitor is desferrioxamine mesylate, a haem derivative, penicillamine, tiopronin, trientine dihydrochloride, diethyldithiocarbamate, disodium/trisodium edetate, acetylsalicylic acid, edetic acid, unithiol, a tocopherol, catechin, mannitol, azithromycin, silidianin or ascorbic acid.

5. A method according to claim 1 comprising determining abzyme activity in the vascular system of said individual prior to administration of said bacterial composition.

6. A method according to claim 1 wherein said bacterial composition comprises one or more enteric bacteria.

7. A method according to claim 1 wherein said bacterial composition comprises one or more of Lactobacilli spp, E. coli and Bifidobacteria spp and Streptococcal spp.

8. A method according to claim 7 wherein said bacterial composition comprises one or more of: L plantarum, L. reuteri, L. bulgaricus Lactobacillus GG, L. acidophilus, L. casel, L. fermentum, L. gasseri, L. johnsonii, L. lactis, L. paracasei L. plantarum, L. rhamnosus and L. salivarius.

9. A method according to claim 8 wherein said bacterial composition comprises L. acidophilus, L. Casei casei, and L. casei rhamnosus.

10. A method according to claim 8 wherein said bacterial composition comprises one or more of: B. bifidum, B. breve, B. lactis, B. longum and B. infantis.

11. A method according to claim 8 wherein said bacterial composition comprises S. thermophilus.

12. A method according to claim 1 wherein said bacterial composition comprises 10⁶ to 10¹⁰ bacterial cells.

13. A method of preventing, delaying or reducing the risk of onset of an atherosclerotic disorder in an individual comprising:
administering a bacterial composition to the individual.

14. A method according to claim 13 wherein the individual has a history of atherosclerotic disorders.

15. A method according to claim 13 wherein the individual has a history of elevated levels of abzymes in the serum.

16. A method according to claim 13 wherein the individual has been treated for an atherosclerotic disorder.

17. A method according to claim 13 comprising determining abzyme activity in the vascular system of the individual prior to administration of the bacterial composition.

18. A method according to claim 13 wherein the individual has normal serum abzyme levels.

19. A method according to claim 13 wherein said bacterial composition comprises one or more of Lactobacilli spp, E. coli and Bifidobacteria spp and Streptococcal spp.

20. A method according to claim 19 wherein said bacterial composition comprises one or more of: L plantarum, L. reuteri, L. bulgaricus Lactobacillus GG, L. acidophilus, L. casei, L. fermentum, L. gasseri, L. johnsonii, L. lactis, L. paracasei L. plantarum, L. rhamnosus and L. salivarius.

21. A method according to claim 20 wherein said bacterial composition comprises L. acidophilus, L. casei casei, and L. casei rhamnosus.

22. A method according to claim 20 wherein said bacterial composition comprises one or more of: B. bifidum, B. breve, B. lactis, B. longum and B. infantis.

23. A method according to claim 20 wherein said bacterial composition comprises: S. thermophilus.

24. A method according to claim 13 wherein said bacterial composition comprises 10⁶ to 10¹⁰ cells.

25. Use of a bacterial composition in the manufacture of a medicament for preventing, delaying or reducing the risk of recurrence of an atherosclerotic disorder in an individual.

26. Use of a bacterial composition in the manufacture of a medicament for treatment of an atherosclerotic disorder in an individual.

27. Use according to claim 25 wherein the individual has a history of atherosclerotic disorders.

28. Use according to claim 25 wherein the individual has previously been treated for an atherosclerotic disorder.

29. Use according to claim 25 wherein the individual has recovered from an atherosclerotic disorder.

30. Use according to claim 25 wherein the individual has a history of elevated levels of abzymes in the serum.

31. Use according to claim 25 wherein the individual has elevated levels of abzymes in the serum.

32. Use according to claim 25 wherein the individual has normal serum abzyme levels.

33. Use according to claim 25 wherein said bacterial composition comprises one or more of Lactobacilli spp, E. coli and Bifidobacteria spp and Streptococcal spp.

34. Use according to claim 33 wherein said bacterial composition comprises one or more of: L plantarum, L. reuteri, L. bulgaricus Lactobacillus GG, L. acidophilus, L. casei L. fermentum, L. gasseri, L. johnsonii, L. lactis, L. paracasei L. plantarum, L. rhamnosus and L. salivarius.

35. Use according to claim 34 wherein said bacterial composition comprises L. acidophilus, L. casei casei, and L. casei rhamnosus.

36. Use according to claim 33 wherein said bacterial composition comprises one or more of: B. bifidum, B. breve, B. lactis, B. longum and B. infantis.

37. Use according to claim 33 wherein said bacterial composition comprises S. thermophilus.

38. Use according to claim 25 wherein said bacterial composition comprises 10⁶ to 10¹⁰ cells.
